(19) United States
(12) Patent Application Publication
(10) Pub. No.: US 2010/0179658 A1
(43) Pub. Date: Jul. 15, 2010

(54) Vertebral Body Cage

(75) Inventors: Thomas B. Freeman, Tampa, FL (US); Wesley M. Johnson, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/730,839

(22) Filed: Mar. 24, 2010

Related U.S. Application Data
(63) Continuation-in-part of application No. PCT/US08/77504, filed on Sep. 24, 2008.
(60) Provisional application No. 60/974,630, filed on Sep. 24, 2007.

(51) Int. Cl. A61F 2/44

(52) U.S. Cl. 623/17.12; 623/17.11; 623/17.16

(57) ABSTRACT

A intervertebral body device for supporting adjacent vertebrae includes a vertebral body member having at least one containment portion and one communicating portion. The containment portion and communication portion provide two distinct functional regions within the cage. One region of the cage provides for the use of a biologic material, and the region of the cage provides holes in the side or the front of the cage in order to allow bony ingrowth into that portion of the cage. Where numerous segments are needed, these components can be arranged in any combination.

### CROSS REFERENCE TO RELATED APPLICATIONS

This application is a continuation in part of prior filed International Application, Serial Number PCT/US08/77504 filed Sep. 24, 2008 which claims priority to U.S. Provisional Application No. 60/974,630 filed Sep. 24, 2007, which is incorporated herein by reference.

### BACKGROUND OF THE INVENTION

Vertebral bodies are removed for a variety of reasons, including degenerative disease, tumors, and trauma, among others. After the vertebral bodies are removed, the spine must be reconstructed to maintain spinal alignment and stability so that the patient can be mobilized. Currently a variety of types of cages are utilized, with variations in height, shape, materials, and end plates, among other variations. However, all cages have one of two intrinsic characteristics: either the outer portions of the cage are solid, acting as a containment device, or a mesh is utilized, allowing bone in-growth from the adjacent vertebral body into bone within the cage.

When biologics such as bone morphogeneic protein are utilized, it is necessary to use a containment design in order to prevent dissemination of bone morphogeneic protein into regions where fusion is not desired, for example along the dura or in soft tissues of the neck. Conversely, it is desirable to have a cage where biologics such as bone morphogeneic protein can be utilized, which also allow bone ingrowth from the side of the vertebral body without losing the containment characteristic of the cage necessary to contain the bone morphogeneic protein. Current cage designs utilize either exclusively containment design or a design that allows for bony ingrowth.

Interbody cages are used in the cervical, thoracic and lumbar spine after removal of vertebral bodies for a variety of reasons. It is desirable in some circumstances to fill the cages with biologics including, but not limited to, bone morphogenetic protein. In this circumstance it is desirable to have a containment design to limit the bone morphogenetic protein or other biologic from being exposed to dura or soft tissues, or to minimize ectopic bone growth. However, it is also desirable to have a design for a cage that allows ingrowth of the bone from the sides or front of the vertebral body to help stabilize the cage once fusion occurs, in addition to the end-to-end bony fusion through the end caps of the cage. Such holes in the side of the cage are not compatible with containment of biologic material such as BMP.

### SUMMARY OF INVENTION

The embodiments disclosed herein utilize both containment regions and ingrowth regions in the same cage. Bony ingrowth from the sides gives greater stability to the cage. The invention allows both containment of biologics in one or more region(s) of the cage and ingrowth of bone through a noncontainment portion of the cage. Therefore the invention provides the simultaneous ability to have the sides of the cages (vs. ends) restrain biologics through a containment design, and simultaneously allow bone ingrowth into the sides of the cage in noncontainment regions of the cage.

The invention can be used in the cervical, thoracic or lumbosacral spine. The invention can be inserted anteriorly, laterally, transversally or through other approaches known in the art. Furthermore, the invention can be used with bone morphogeneic protein, bone morphogeneic protein plus local or other autologous bone, BMP plus any carrier including, but not limited to bone bank or other allograft bone, or any other combination of bone and biologic material, as well as any bone substitute which is of common practice or potential future practice to those knowledgeable in the art. Such devices can be made of any biocompatible material where appropriate strength is available. Ideally, such a material would match the structural characteristics of the adjacent vertebral bodies as much as possible.

In one embodiment, the invention includes fixed-length, “stackable” subassemblies of at least one containment cage and one communication cage. In an alternate embodiment, the invention includes an integral device having at least one containment region and one communication region. In yet another embodiment, the invention includes an expandable cage having at least one communication region and one containment region. The shape of the cage (round, rectangular, etc.) and end plate shape (flat, lordotic, convex, etc.) can be any shape in common practice to those knowledgeable in the art.

### BRIEF DESCRIPTION OF THE DRAWINGS

For a fuller understanding of the invention, reference should be made to the following detailed description, taken in connection with the accompanying drawings, in which:

FIG. 1A is a perspective view of an integrated intervertebral body device according to a first embodiment of the invention.

FIG. 1B is an anterior view of an integrated intervertebral body device according to a first embodiment of the invention.

FIG. 1C is a lateral view of an integrated intervertebral body device according to a first embodiment of the invention.

FIG. 2 is a sem-exploded, anterior view of a stackable intervertebral body device according to a second embodiment of the invention.

FIG. 3 is an elevated perspective view of a containment cage.

FIG. 4 is an elevated perspective view of a communication cage.

FIG. 5A is an anterior view of an illustrative communication cage having a serrated bearing surface.

FIG. 5B is an anterior view of an illustrative communication cage having a lordotic bearing surface.

FIG. 6 is a lateral view of the inventive device implanted in a resected vertebral space.

FIG. 7 is a perspective view of an expandable intervertebral body device according to a third embodiment of the invention.

FIG. 8 is a perspective view of an alternate expandable intervertebral body device according to a third embodiment of the invention.

### DETAILED DESCRIPTION OF THE PREFERRED EMBODIMENT

According to a first embodiment, the invention includes an intervertebral body cage having a plurality of functional regions; containment regions and “communication” regions. Containment regions hold therapeutic biologics, such as BMP. The communication regions allow bony in-growth into that portion of the cage. Although the invention includes two types of regions, the invention can be used in limitless configurations as any number of these components can be arranged, i.e. stacked, in any combination. The containment region can be located either at the ends of the device with a communication region in the middle portion of the cage, or visa versa (with mesh on the extremities of the cage and the containment region in the middle). In another embodiment, an expandable cage system provides the same functionality.

The containment region further includes a containment area adapted to hold a suitable osteogenic material or composition. Such osteogenic material includes, for example, autograft, allograft, xenograft, demineralized bone, synthetic and natural bone graft substitutes, such as bioceramics and polymers, and osteoinductive factors. The bony material is placed within the containment area and can be pre-packed into the containment area before the device is implanted, or can be pushed through a plurality of wall openings after the device is in position in the spinal column.

A separate carrier to hold the materials within the containment area can also be used. These carriers can include collagen-based carriers, bioceramic materials, such as BIOGLASS®, hydroxyapatite and calcium phosphate compositions. The carrier material can be provided in the form of a sponge, a block, folded sheet, putty, paste, graft material or other suitable form. Moreover, the osteogenetic compositions contained within the vertebral replacement device can comprise an effective amount of a bone morphogenetic protein, transforming growth factor .beta.1, insulin-like growth factor 1, platelet-derived growth factor, fibroblast growth factor, LIM mineralization protein (LMP), and combinations thereof or other therapeutic or infection resistant agent, held within a suitable carrier material.

The communication region allows bony in-growth through the sides of the cage. Typically, bone in-growth is not likely to originate anteriorly (where soft tissues are found, such as the esophagus) or posteriorly (where the thecal sac is located); yet it is possible to provide communication in these directions if the particular needs of a patient so-require. Generally, however, communication is not needed in the anterior or posterior regions of the device. Use of solid walls anteriorly and posteriorly in the communication cage allows bony in-growth from the sides, where the mesh is located, yet provides increased structural stability. Alternatively, if the device is placed from a lateral approach in the thoracic or lumbar spine, communication may only be needed anteriorly and laterally to facilitate bone ingrowth yet maintain increased strength with a solid wall adjacent to the spinal canal.

The invention, including its subcomponents, can be made in a variety of sizes, as will be appreciated by those of skill in the art. For example, single level corpectomies in the cervical spine range in size from approximately 21 millimeters to 29 millimeters, but generally are between 24 and 27 millimeters in length. Thoracic and lumbar corpectomies have progressively larger heights. The sizes of these cages will therefore reflect the sizes of the spine to be reconstructed. For example, in the cervical spine, cages that are roughly 12 millimeters in height and 15 millimeters in width, appropriately shaped, would be expected to fit into the vast majority of vertebral bodies. Making the device 13-14 millimeters in height may allow greater surface area and placement of more bone within the containment area, but may not always be appropriate in smaller vertebral bodies, for example in the C3 or C4 region in a female where such vertebral bodies may be smaller. However, it is possible that any size which is desired could be manufactured under this embodiment.

A variety of shapes, as well as end plate designs, is also contemplated. Cage width, length, height, shape and endplate shape and footprint design can be any design known or anticipated. For example, the ends of the device may be ridged, flat, lordotic, concave or convex to reflect the anatomy of the vertebral bodies adjacent to the cage.

Any one or all of the components of the vertebral replacement devices can be made from any biocompatible material, including synthetic or natural autograft, allograft or xenograft tissues, and can be resorbable or non-resorbable in nature. Examples of tissue materials include hard tissues, connective tissues, demineralized bone matrix and combinations thereof. Examples of resorbable materials are polylactide, polyglycolide, tyrosine-derived polycarbonate, polyanhydride, polyorthoester, polyphosphazene, calcium phosphate, hydroxyapatite, bioactive glass, and combinations thereof. Examples of non-resorbable materials are non-reinforced polymers, carbon-reinforced polymer composites, PEEK and PEEK composites, shape-memory alloys, titanium, titanium alloys, cobalt chrome alloys, stainless steel, ceramics and combinations thereof and others as well.

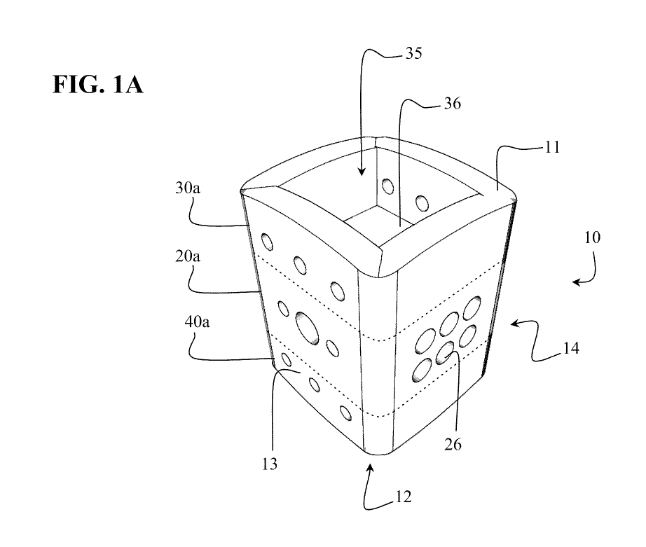

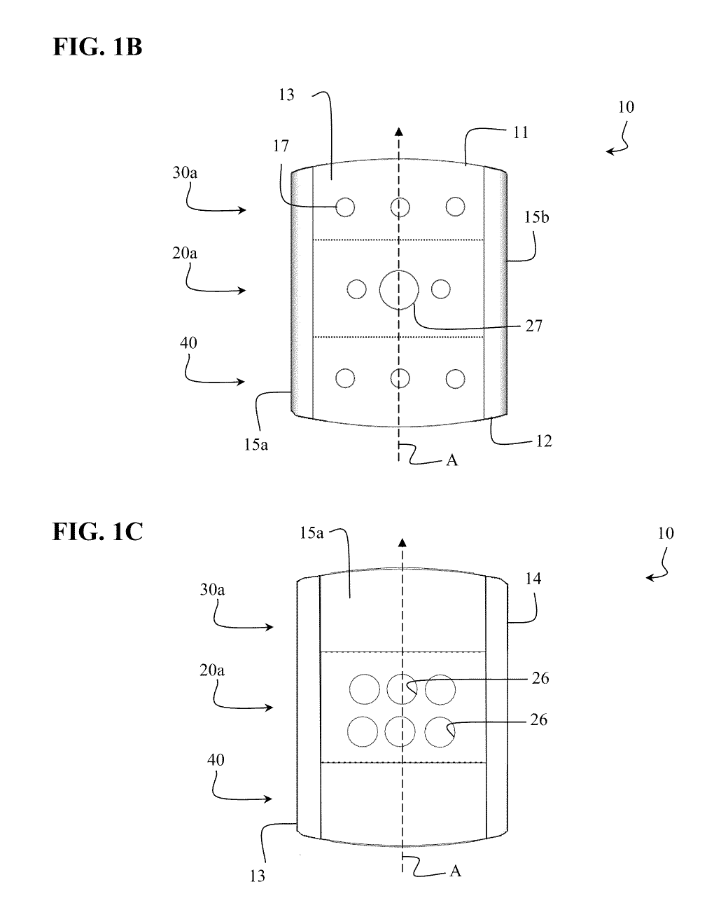

Intervertebral body device 10 is shown in FIGS. 1A-1C as an integral device having a plurality of functional regions. In this embodiment, device 10 includes cranial end 11, caudal end 12, anterior side 13, posterior side 14 and lateral sides 15a and 15b. The various regions of device 10 are delineated by dotted lines. The regions shown include a single communication region 20a sandwiched between upper containment region 30a and lower containment region 40a. Device 10 is illustrated as having a substantially rectangular form that extends along a longitudinal axis A. Other shapes for device 10 are also contemplated, including oval shapes, circular shapes, polygonal shapes and/or any shape known to those of skill in the art.

Containment region 20a further comprises an interior chamber, discussed further below. Communication with the interior of containment region 20a is provided via communication apertures 26 formed in lateral sides 15a and 15b. Containment regions 30a and 40a include containment chambers (35) which are adapted to hold an osteogenetic material, as discussed above. It can be seen in the embodiments of FIGS. 1A-1C that cranial end 11 and caudal end 12 are lordodically shaped.

As shown in FIGS. 1A-1C, device 10 has an outer surface that defines a substantially cross section transverse to longitudinal axis A. Other cross-sectional shapes are also contemplated, including, for example, circular cross-sections and non-circular cross-sections, such as oval, triangular, square, kidney shaped, polygonal, boomerang shaped, D-shaped, or racetrack shaped cross-sections. In the illustrated embodiment, communication region 20a has the same cross-sectional shape as the upper and lower containment regions 30a, 40a to provide a vertebral replacement body device of uniform cross-sectional shape and size along its height.

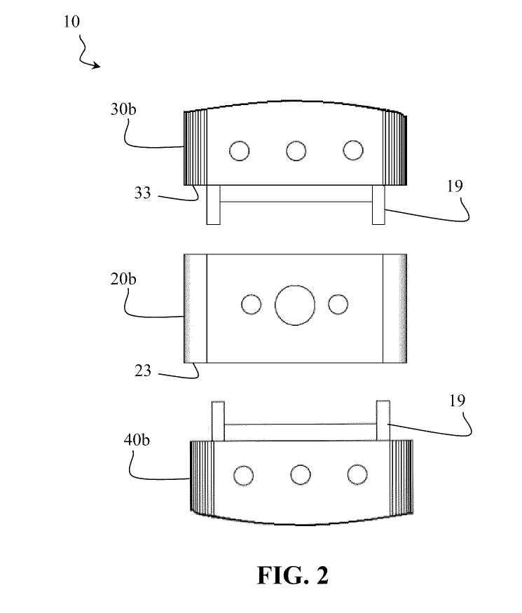

Referring now to FIG. 2, it can be see that the containment region and communication regions can be isolated in individual, but cooperative, sub-assemblies (or “cages”). According to the embodiment of FIG. 2, vertebral body device 10 includes communication cage 20, upper containment cage 30, and a lower containment cage 40.

Upper containment cage 30b and lower containment cage 40b are connected to respective ends of communication cage 20b to provide vertebral body device 10. Upper containment cage 30b is advanced over communication cage 20b so that extension 19 on lower end 33 extends into the communication chamber of communication cage 20b. The engagement of upper containment cage 30b to communication cage 20b resists movement of upper containment cage 30b away from communication cage 20b along axis A (FIGS. 1B and 1C). Lower containment cage 40b is secured to lower end 23 of communication cage 20b in a similar manner.

Axial rotation of upper containment cage 30b relative to communication cage 20b is resisted by the interface between extension 19 of upper containment cage 30b and the respective inner wall surface of containment cage 20b.

Upper and lower containment cages 30b, 40b are illustrated in FIG. 2 as being identical, although it is also contemplated that upper containment cage 30b and lower containment cage 40b can be provided with different configurations and/or sizes.

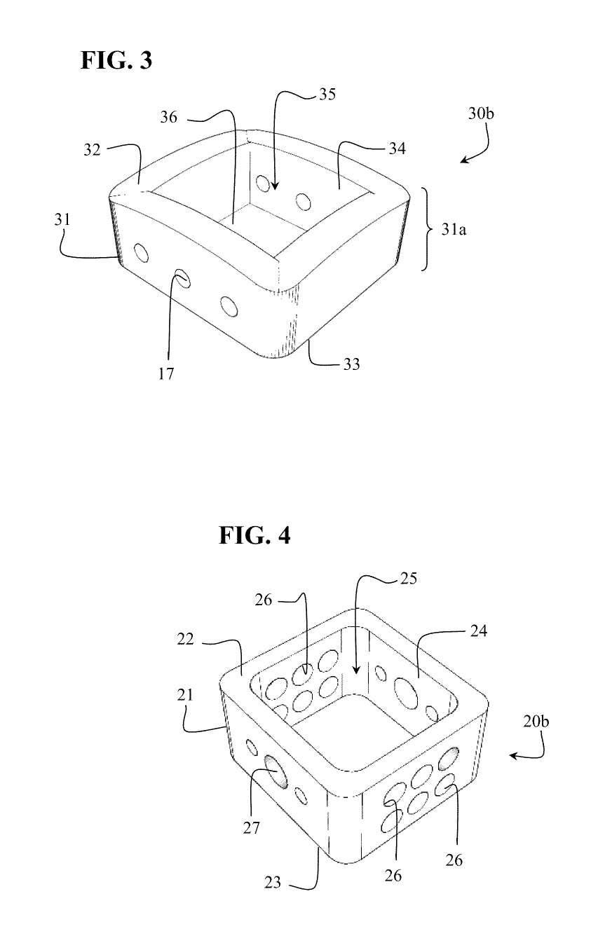

With respect to FIG. 3, only upper containment cage 30b will be further described, it being understood that lower containment cage 40b can be provided with similar features. Upper containment cage 30b includes body 31 extending between upper end 32 and lower end 32. Body 31 has a height 31a between the upper and lower ends 32, 33. Height 31a can be selected so that upper containment cage 30 fits within an inter-vertebral disc space between adjacent vertebrae. Containment cage 30b has an inner wall surface 34 defining a chamber 35 that extends between upper and lower ends 32, 33. Chamber 35 is open at upper end 32 and terminates at lower wall 36. Bone growth can occur through the open end of chamber 35 for fusion between the vertebral bodies supported at each end of device 10.

In an illustrative embodiment, the containment cage may be approximately 9-12 millimeters in length in order to allow a biologic (for example a sponge filled with BMP (Medtronic) or OP-1 (Stryker) 1-mixed with a carrier, among others), plus bone or bone substitute adjacent to it to reduce leakage of BMP into regions adjacent to the cage. In this example, the BMP-sponge, putty or other biologic is expected to occupy a distance of approximately six millimeters. Therefore, the containment-area of the cage will be 10-11 millimeters in height allowing for approximately four millimeters of bone to be placed adjacent to the biologic. It is also contemplated that less bone can be placed medial to the biologic, making the containment cage a shorter construct. Therefore, containment walls of the cage would occupy between 9 and 12 millimeters on each end in an illustrative embodiment. Larger or smaller containment regions can be used if, for example, more BMP is utilized or if more bone or bone substitute or other material is needed to contain the BMP. Containment walls could therefore range from 6 to 20 mm. in length.

Containment cage 30b is provided with a number of alignment holes 37 extending at least partially there through from the exterior surface of body 31. Holes 37 can be threaded or otherwise sized and/or configured for engagement with one or more insertion instruments.

Communication cage 20b, shown in FIG. 4, further includes a body (21) extending between an upper end 22 and an opposite lower end 23. Communication cage 20 has an inner wall surface 24 that defines chamber 25 extending between and opening at the outer ends upper end 22 and an opposite lower end 23. The wall of body 21 includes a number of substantially circular communicating apertures 26 which extend through the wall and communicate with chamber 25. Other shapes for communicating apertures 26 are also contemplated, including non-circular shapes such as a square, diamond, oval and/or rectangular shapes, circular shapes, and/or polygonal shapes. The wall of body 21 also includes a number of alignment holes 27 extending at least partially there through. Alignment holes 27 can be threaded or otherwise sized and/or configured for engagement with one or more insertion instruments (not shown.)

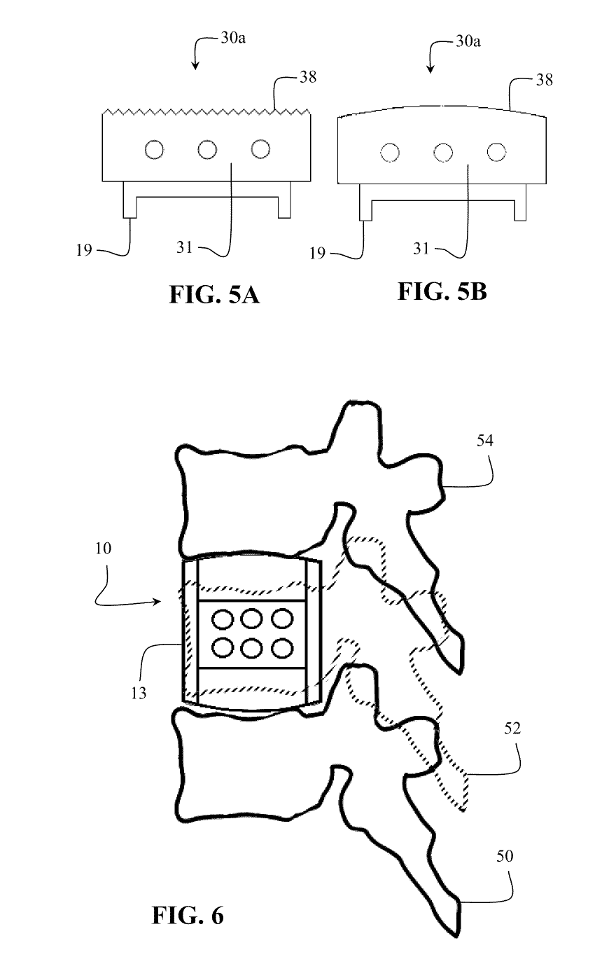

Device 10 further includes a number of bearing surfaces spaced around the upper and lower surfaces. Bearing surface 38, shown in FIGS. 5A and 5B on the upper surface of containment cage 30b, provides surface area contact between the upper end of body 31 and the adjacent vertebral endplate, providing frictional resistance to body 31 sliding or twisting relative to the adjacent vertebral endplate. Bearing surface 38 comprises a plurality of teeth-like structures which are substantially triangular in cross-section (lordodic), as shown in the illustrated embodiment of FIG. 5A. Other shapes are also contemplated, for example FIG. 5B shows a lordotic bearing surface (38). It is further contemplated that device 10 could be provided with a single, continuous bearing surface extending completely around the upper surface thereof.

It is also contemplated that communication cage 20b could be provided with one end configured to bear against a vertebral endplate, and that only one of the upper and lower containment cages 30b, 40b is engaged to the other end of communication cage 20b. The assembled device could then be placed between adjacent vertebrae with an end of communication cage 20b and an end of the selected upper or lower containment cage 30b, 40b in contact with the adjacent vertebral endplates.

Device 10 can be used to replace a vertebra that has been removed from the spinal column segment using known techniques. Device 10 is assembled by securing upper containment cage 30b to one end of communication cage 20b and securing lower containment cage 40b to the other end of communication cage 20b. This provides a vertebral replacement device 10 that has an overall height that is equal to the sum of the heights of communication cage 20b, upper containment cage 30b, and lower containment cage 40b.

As shown in FIG. 6, the vertebral body device 10 can be placed between vertebra 50 and vertebra 54 after removal of vertebra 52. Replacement of more than one vertebra is also contemplated. FIG. 6 illustrates placement of device 10 in the disc space between adjacent vertebrae 50, 54 to function as an inter-body fusion device.

In another embodiment, the invention includes an expandable inter-vertebral device. In this embodiment, any mechanism standard in the art for expansion of a cage can be utilized. Illustrative devices include ratcheting and dialing mechanism, among others.

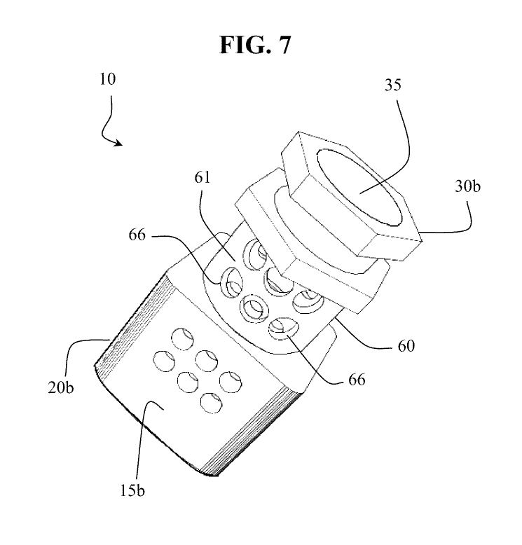

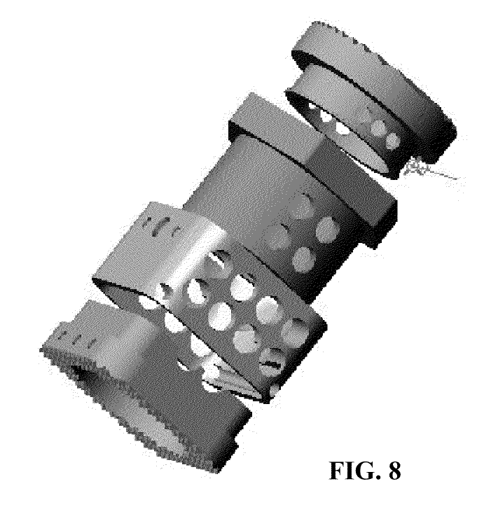

FIG. 7 illustrates an illustrative expandable cage. In this embodiment, inter-vertebral body device 10 includes communication cage 20b having a bearing surface on the caudal end thereof, expanding cage 60 and containment cage 30b. In this embodiment, expanding cage 60 is telescopically disposed within communication cage 20b. As it can be seen, expanding cage 60 has a plurality of communicating apertures 66 formed in sidewall 61 thereof. Communicating apertures 66 maintain communication with communication chamber inside communication cage 20b, communication apertures 26 (formed in lateral sides 15a and 15b of communication cage 20b ) and the exterior of the device 10.

It will also be appreciated that the arrangement of FIG. 7 can be altered such that a containment cage receives expanding cage 60. The side wall of expanding cage 60 would preferably not contain communication apertures in such an arrangement.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A vertebral replacement device, comprising:
a cranial end and a caudal end, wherein at least one of the cranial end and caudal end further comprises an end surface for engaging an endplate of an adjacent vertebral body;
at least one containment region further comprising a body extending between a first end and an opposite second end, and an inner wall that extends between the first and second ends, thereby defining a containment area; and
at least one communication region further comprising a body extending between a first end and an opposite second end, and an inner wall surface extending between the first and second ends thereby defining a communication chamber.

2. The device of claim 1, wherein the containment region is disposed at the cranial end of the device.

3. The device of claim 1, wherein the communication region is disposed at the cranial end of the device.

4. The device of claim 1, wherein the containment region is disposed at the caudal end of the device.

5. The device of claim 1, wherein the communication region is disposed at the caudal end of the device.

6. The device of claim 1, further comprising an anterior side, a posterior side, a first lateral side and a second lateral side.

7. The device of claim 1, wherein the containment region further comprises a transverse wall disposed at the second end of the containment region body.

8. The device of claim 7, wherein the containment area is open at the first end of the containment region body and terminates at the transverse wall.

9. The device of claim 1, wherein the containment area is adapted to receive a bone growth material.

10. The device of claim 9, wherein the bone growth material includes one or more selected from group consisting of: bone morphogenetic protein, transforming growth factor .beta.1, insulin-like growth factor 1, platelet-derived growth factor, fibroblast growth factor, and LIM mineralization protein (LMP).

11. The device of claim 10, wherein said bone growth material is provided in a carrier having a form selected from the group consisting of: a sponge, a block, folded sheet, putty, and paste.

12. The device of claim 1, wherein the communication chamber is open at the first and second ends of the communication region body.

13. The device of claim 1, further comprising:
a posterior side, an anterior side, a first lateral side and a second lateral side; and
a communication area in at least one side of the communication region body between the communication chamber and at least one side of the device.

14. The device of claim 13, wherein the communication area is in at least one lateral side of the communication region body.

15. The device of claim 13, wherein the communication area comprises a plurality of apertures extending from the communication chamber through the communication region body.

16. The device of claim 15, wherein the apertures extend through at least one lateral side of the communication area body.

17. The device of claim 13, wherein the communication area is a mesh.

18. The device of claim 1, wherein one of the first and second ends of the containment region is positionable adjacent at least one of the first and second ends of the communication region.

19. The device of claim 1, wherein one of the first and second ends of the communication region is positionable adjacent at least one of the first and second ends of the containment region.

20. The device of claim 1, wherein one of the first and second ends of the containment region is integrally formed with at least one of the first and second ends of the communication region.

21. The device of claim 1, wherein one of the first and second ends of the communication region is integrally formed at least one of the first and second ends of the containment region.

22. The device of claim 1, further comprising an engaging member, having a first end and an opposite second end, extending between the containment region and the communication region.

23. The device of claim 22, wherein the first end of the engaging member is connected to the first or second end of the containment region, and wherein the second end of the engaging member is received in the first or second ends of the communication chamber.

24. The device of claim 22, wherein the first end of the engaging member is connected to the first or second end of the communication region, and wherein the second end of the engaging member is received in the containment area.

25. The device of claim 1, further comprising a second containment region; wherein the communication region is disposed between the first and second containment regions.

26. The device of claim 1, further comprising a second communication region; wherein the containment region is disposed between the first and second communication regions.
